(19) United States
(12) Patent Application Publication
(10) Pub. No.: US 2016/0362442 A1
(43) Pub. Date: Dec. 15, 2016

(54) Method For DHEA Enanthate Processing

(71) Applicant: IronMag Labs, Henderson, NV (US)

(72) Inventor: Robert DiMaggio, Henderson, NV (US)

(21) Appl. No.: 14/736,103

(22) Filed: Jun. 10, 2015

(51) Int. Cl. C07J 1/00

(52) U.S. Cl. CPC C07J 1/0011

(57) ABSTRACT

A method is provided for producing dehydroepiandrosterone (DHEA) enanthate. The method comprises placing a portion of unprocessed DHEA into a solvent, such as Benzene, and allowing the DHEA to dissolve completely into the solvent, thereby forming a solution. The unprocessed DHEA preferably comprises unprocessed 5-DHEA, 1-DHEA, or 4-DHEA. A suitable quantity of Pyridine is then mixed into the solution. Heptanoyl Chloride is added slowly into the solution to cause a reaction of the unprocessed DHEA. Once substantially all of the unprocessed DHEA has been reacted, the addition of Heptanoyl Chloride is ceased. An effective amount of the DHEA enanthate may be administered to a human so as to improve at least one of adrenal hormonal balance, improved immune system function, reduction of adipose tissue, skeletal muscle growth, reduction of abdominal bloating, improved recovery from training, increase in strength, and increased endurance.

### FIELD

The field of the present disclosure generally relates to dietary supplements. More particularly, the field of the present disclosure relates to a method for producing dehydroepiandrosterone (DHEA) enanthate isomers for human supplementation to improve human physical performance.

### BACKGROUND

The adrenal gland produces many steroid hormones. These steroid hormones play a major role in many body processes including, for example, skeletal muscle growth, red blood cell production (erythropoiesis), regulation of glucose and insulin levels, and cellular aging. The steroids produced by the adrenal gland can be divided into three groups: glucocorticoids, which influence carbohydrate metabolism; mineralocorticoids, which control electrolyte and water balance; and sex steroid hormones.

Glucocorticoids such as cortisol regulate catabolism of skeletal muscle proteins into amino acids. These amino acids are then transported to the liver and converted into glucose during gluconeogenesis. Excessive amounts of glucocorticoids can result in higher blood glucose and insulin levels and can contribute to increased body fat and type II diabetes. Glucocorticoids are also known to play a role in the aging process by increasing cellular and mitochondrial breakdown. Mineralocorticoids such as aldosterone help the body to retain sodium and water. Excesses of mineralocorticoids can result in hypertension and cardiovascular disease. The sex steroid hormones include adrenal androgens and DHEA. Adrenal androgens oppose the actions of glucocorticoids and result in skeletal muscle anabolism (the opposite action of catabolism), reductions in blood glucose and insulin levels, reduction in body fat, and are believed to decrease the rate of cellular aging and increased red blood cell production. DHEA production by the adrenal glands is known to decline markedly as aging progresses.

With normal younger adults, all three groups of adrenal steroids are produced in a healthy balance. As people age, however, less DHEA is produced resulting in relatively greater amounts of glucocorticoids and mineralocorticoids, resulting in a disruption of a healthy hormone balance.

DHEA supplementation is believed to be useful in treatment of aging and obesity, and to stimulate erythropoiesis and skeletal muscle anabolism. In addition, supplemental DHEA may help restore the balance of adrenal steroids.

DHEA is commonly used as a dietary supplement. Unfortunately, DHEA is rapidly metabolized by liver enzymes, such as sulfotransferases. Sulfotransferases rapidly convert much of the supplementary DHEA into DHEA sulfate, which is quickly excreted from the body and is not effective as an anti-aging, muscle-building or fat reduction compound. In addition, DHEA sulfate does not restore the balance of the adrenal steroids discussed above. As a result, frequent and larger doses of DHEA must be taken to achieve a desired result.

DHEA is also metabolized in the body to one of several compounds including, for example, etiocholanolone (5-beta-androstan-3-alpha-ol-17-one), beta etiocholanolone (5-beta-androstan-3-beta-ol-17-one), androsterone (5-alpha-androstan-3-alpha-ol-17-one), epiandrosterone (5-alpha-androstan-3-beta-ol-17-one), 7-keto-DHEA, 7-alpha-hydroxy-DHEA, 7-beta-hydroxy-DHEA, androstenedione, estrone, and estradiol.

There is great individual variability in the metabolism of oral DHEA. The DHEA metabolites estrone and estradiol can result in negative estrogenic side effects for males including growth of male breast tissue, known as gynecomastia. Some individuals have poor bioavailability of DHEA as a result of sulfation in the liver, and large doses must be taken to elicit any desired effects. These increased doses of DHEA can result in increased conversion to estrone and estradiol, with resulting negative side effects.

What is needed, therefore, are compounds which provide the beneficial effects of high DHEA levels in the body for extended periods of time, yet reduce the undesired DHEA side effects discussed above.

### SUMMARY

A method is provided for producing dehydroepiandrosterone (DHEA) enanthate. The method comprises placing a portion of unprocessed DHEA into a solvent, such as Benzene, and allowing the DHEA to dissolve completely into the solvent, thereby forming a solution. The unprocessed DHEA preferably comprises 5-DHEA, 1-DHEA, or 4-DHEA. A suitable quantity of Pyridine is then mixed into the solution. Heptanoyl Chloride is added slowly into the solution to cause a reaction of the unprocessed DHEA. Once substantially all of the unprocessed DHEA has been reacted, the addition of Heptanoyl Chloride is ceased. An effective amount of the DHEA enanthate may be administered to a human so as to improve at least one of adrenal hormonal balance, improved immune system function, reduction of adipose tissue, skeletal muscle growth, reduction of abdominal bloating, improved recovery from training, increased strength, and increased endurance. In some embodiments, the DHEA enanthate may be administered by way of any one of perorally, transdermally, sublingually, and instranasally. In some embodiments, the effective amount is a daily dosage ranging between 10 mg and 2000 mg.

In an exemplary embodiment, a method for producing dehydroepiandrosterone (DHEA) enanthate comprises placing a portion of unprocessed DHEA into a solvent; allowing the portion of unprocessed DHEA to dissolve completely into the solvent, thereby forming a solution; mixing a first fluid into the solution; adding a second fluid slowly into the solution to cause a reaction of the portion of unprocessed DHEA; and ceasing adding the second fluid once substantially all of the portion has been reacted.

In another exemplary embodiment, the solvent comprises Benzene. In another exemplary embodiment, the first fluid comprises Pyridine. In another exemplary embodiment, the second fluid comprises Heptanoyl Chloride. In another exemplary embodiment, the portion of unprocessed DHEA comprises 5-DHEA. In another exemplary embodiment, the portion of unprocessed DHEA comprises 1-DHEA. In another exemplary embodiment, the portion of unprocessed DHEA comprises 4-DHEA.

In another exemplary embodiment, the method further comprises administering an effective amount of the DHEA enanthate to a human so as to improve at least one of adrenal hormonal balance, improved immune system function, reduction of adipose tissue, skeletal muscle growth, reduction of abdominal bloating, improved recovery from training, increase in strength, and increased endurance. In another exemplary embodiment, administering is selected from the group consisting of perorally, transdermally, sublingually, and instranasally. In another exemplary embodiment, the effective amount is a daily dosage ranging between 10 mg and 2000 mg.

In an exemplary embodiment, a method for providing dehydroepiandrosterone (DHEA) enanthate to a human so as to improve at least one of adrenal hormonal balance, improved immune system function, reduction of adipose tissue, skeletal muscle growth, reduction of resultant abdominal bloating, improved recovery from training, increase in strength, and increased endurance comprises dissolving a portion of unprocessed DHEA into a suitable quantity of Benzene to form a solution; adding an effective amount of Pyridine into the solution; causing a formation of the DHEA enanthate by slowly adding Heptanoyl Chloride into the solution until substantially all of the portion of unprocessed DHEA has been reacted; and administering an effective amount of the DHEA enanthate to the human.

In another exemplary embodiment, the method further comprises providing the DHEA enanthate in the form of any one of 5-DHEA enanthate, 1-DHEA enanthate, and 4-DHEA enanthate. In another exemplary embodiment, administering is selected from the group consisting of perorally, transdermally, sublingually, and instranasally.

### BRIEF DESCRIPTION OF THE DRAWINGS

The drawings refer to embodiments of the present disclosure in which:

FIG. 1 is a chemical formula illustrating an exemplary embodiment of a process for producing 5-DHEA enanthate, according to the present disclosure;

FIG. 2 is a chemical formula illustrating an exemplary embodiment of a process for producing 1-DHEA enanthate in accordance with the present disclosure; and

FIG. 3 is a chemical formula illustrating an exemplary embodiment of a process for producing 4-DHEA enanthate, according to the present disclosure.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the invention disclosed herein may be practiced without these specific details. In other instances, specific numeric references such as “first solution,” may be made. However, the specific numeric reference should not be interpreted as a literal sequential order but rather interpreted that the “first solution” is different than a “second solution.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In general, the present disclosure describes a method for providing dehydroepiandrosterone (DHEA) enanthate to a human so as to improve at least one of adrenal hormonal balance, improved immune system function, reduction of adipose tissue, skeletal muscle growth, reduction of abdominal bloating, improved recovery from training, increased strength, and increased endurance. The method comprises dissolving a portion of unprocessed DHEA into a suitable quantity of Benzene to form a solution. An effective amount of Pyridine is added into the solution. A formation of the DHEA enanthate is caused by slowly adding Heptanoyl Chloride into the solution until substantially all of the unprocessed DHEA has been reacted. The DHEA enanthate preferably is comprised of any one of 5-DHEA enanthate, 1-DHEA enanthate, and 4-DHEA enanthate.

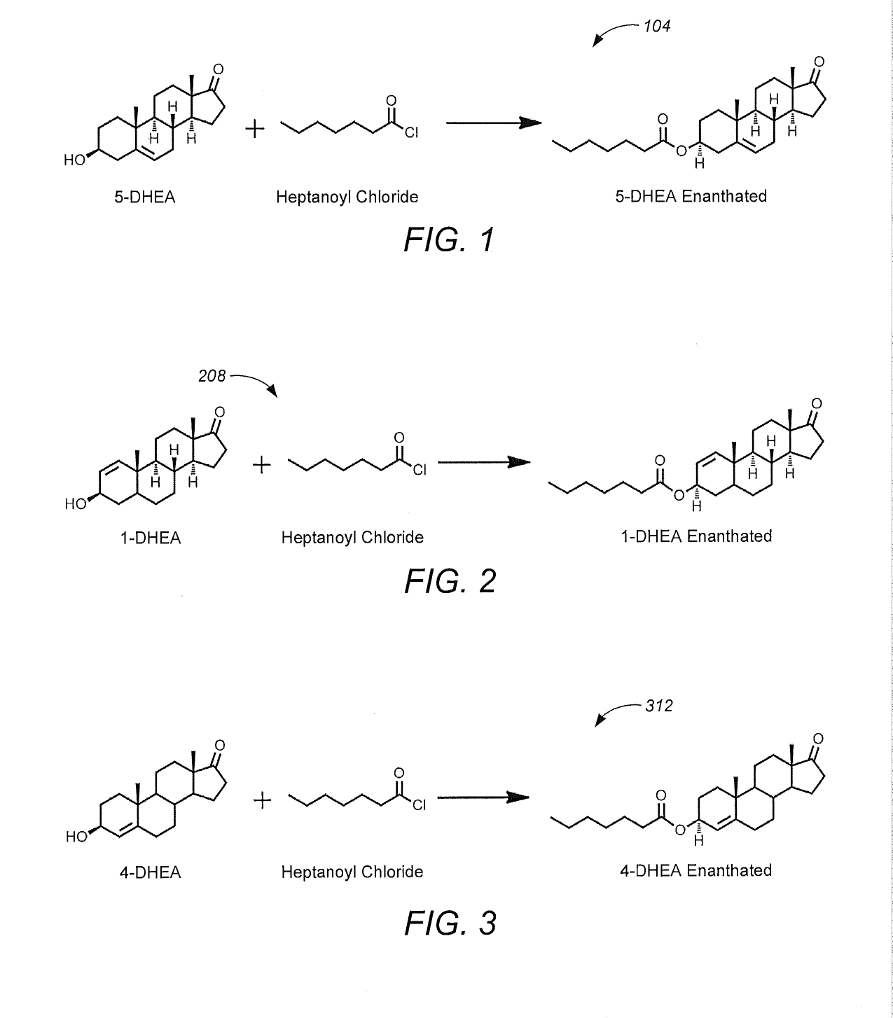

FIG. 1 is a chemical formula illustrating an exemplary embodiment of a process 104 for producing 5-DHEA enanthate, according to the present disclosure. The process 104 begins by placing a desired portion of unprocessed DHEA, in raw material form into a solvent and allowing the DHEA to completely dissolve into the solvent. Preferably, the solvent comprises Benzene. Once the DHEA is suitably dissolved into the solvent, a first fluid comprising Pyridine may be mixed into the solution. A reaction of the portion of unprocessed DHEA may be caused by adding a second fluid comprising Heptanoyl Chloride to the solution. As will be appreciated, the Heptanoyl Chloride preferably is added slowly, such as by way of adding drops to the solution, so as to allow all of the unprocessed DHEA to react. As indicated in FIG. 1, reaction of the solution with the Heptanoyl Chloride produces 5-DHEA enanthate. As will be appreciated, although 5-DHEA enanthate produced by way of the process 104 has a double bond located in the 5th position, 5-DHEA enanthate may be considered to comprise “generic” or regular DHEA.

Although DHEA has been popular since the 1980s due to an ability to increase energy and wellbeing when taken at a dosage of between 50 and 100 mg/day, more recently higher dosages have become popular in an attempt to improve body composition. It will be recognized that higher dosages of DHEA generally give rise to mild anabolic and thermogenic effects, making DHEA popular among trainees attempting to reduce body fat by way of training and dieting. Supplementation with DHEA has been shown to exhibit good muscle sparing, anti-catabolic properties during calorie deficient diets, as well as improved exercise endurance and recovery from training. Further, DHEA is associated with improvements in immune system function in trainees.

As is well known, DHEA generally converts to testosterone at a rate of substantially 1%. Further, DHEA exhibits a relatively high conversion rate to 5-androstenediol and 7-oxo-DHEA. The 5-androstenediol produces mild androgenic and anabolic effects associated with DHEA supplementation, while the 7-oxo-DHEA gives rise to thermogenic properties suitable for reducing body fat in trainees. The mild androgenic properties of DHEA rarely produce hair loss or acne. Despite possessing moderate estrogenic properties, DHEA rarely produces gynecomastia or other undesirable phenomena associated with hormone-based supplementation. DHEA supplementation is frequently associated with elevated energy or motivational energy, which in some cases may present as anxiety or sleeplessness in more sedentary trainees. It will be appreciated that the balanced hormonal properties of DHEA provide a wide range of benefits, and thus is advantageous for cutting body fat, as well as keeping muscle gains lean during a body mass building cycle.

FIG. 2 is a chemical formula illustrating an exemplary embodiment of a process 208 for producing 1-DHEA enanthate in accordance with the present disclosure. The process 208 for producing 1-DHEA enanthate is substantially similar to the process 104, illustrated in FIG. 1, with the exception that the process 208 begins by placing a desired portion of unprocessed 1-DHEA, in raw material form, into the solvent and allowing the portion to dissolve completely into the solvent. As mentioned above, the solvent preferably is comprised of Benzene. Next, Pyridine may be mixed into the solution of 1-DHEA and Benzene. Heptanoyl Chloride may then be slowly added to the solution so as to drive the chemical reaction illustrated in FIG. 2. The Heptanoyl Chloride preferably may be added slowly, such as by way of adding drops to the solution, so as to allow all of the unprocessed 1-DHEA to react, thereby producing 1-DHEA enanthate as desired.

As will be recognized, 1-DHEA is a naturally occurring DHEA isomer which does not convert to testosterone or estrogen. Rather, 1-DHEA converts to non-estrogenic 1-testosterone at a rate generally less than 2%. In addition, 1-DHEA is known to convert to 1-androstenediol, which gives rise to potent muscle building due to nitrogen retention, as well as muscle hardening and strength gains. Also associated with 1-androstenediol is a reduction of abdominal bloating, as well as improved recovery from heavy training. Further, 1-DHEA does not activate estrogen receptors, as does DHEA, and thus 1-DHEA is suitable for stacking with other estrogenic steroids, such as by way of non-limiting example, 4-DHEA, so as to promote clean gains in muscle tissue.

As with other DHEA isomers, 1-DHEA is naturally occurring, non-toxic, and exhibits mild side-effects. Temporary side-effects typically include oily skin, reduced fertility, and increased hair shedding. A notable side-effect is a suppression of natural testosterone production, which makes Post Cycle Therapy (PCT) a necessity after running a 1-DHEA cycle. Some trainees have reported a degree of lethargy when supplementing with 1-DHEA; however, stacking 1-DHEA with DHEA or 4-DHEA is well known to decrease perceived lethargy due to supplementing with 1-DHEA alone. Thus, 1-DHEA is generally considered to be a very safe, legal, and effective lean muscle building agent.

FIG. 3 is a chemical formula illustrating an exemplary embodiment of a process 312 for producing 4-DHEA enanthate, according to the present disclosure. The process 312 begins by placing a desired portion of unprocessed 4-DHEA, in raw material form, into a solvent and allowing the 4-DHEA to completely dissolve into the solvent. As mentioned with respect to the processes 104 and 208, the solvent use in the process 312 illustrated in FIG. 3 preferably comprises Benzene. Once the 4-DHEA is suitably dissolved into the solvent, a first fluid comprising Pyridine may be mixed into the solution. A reaction of the portion of unprocessed 4-DHEA may be initiated by introducing Heptanoyl Chloride into the solution. Upon adding the Heptanoyl Chloride slowly, such as by way of adding drops to the solution, the reaction of the solution with the Heptanoyl Chloride produces 4-DHEA enanthate.

As with other DHEA isomers, 4-DHEA is a naturally occurring DHEA isomer, although a double bond in the 4th position dramatically differentiates 4-DHEA from the other isomers. As is well known, 4-DHEA converts to 4-androstenediol, instead of 5-androstenediol, as in the case of 5-DHEA, thereby boosting the anabolic potency of 4-DHEA to more than two times greater than regular DHEA. Similarly, 4-DHEA also exhibits a higher conversion rate to testosterone as compared to regular DHEA, while also lacking calorie burning thermogenic properties, thereby offering superior calorie retention for noticeable gains in strength, recovery from training, lean tissue growth, and weight gain.

Generally, 4-DHEA exhibits a mild estrogen conversion which may be easily balanced with a non-aromatizing steroid such as androsterone or 1-DHEA. As with other DHEA isomers, 4-DHEA is naturally occurring, non-toxic, and exhibits mild side-effects, such as oily skin, reduced fertility, abdominal bloating, and increased hair shedding. A notable side-effect is a suppression of natural testosterone production, which makes PCT a necessity after running a 4-DHEA cycle. As will be appreciated, 4-DHEA is considered to be generally a very safe, legal, and effective lean muscle building agent.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended claims.

1. A method for producing dehydroepiandrosterone (DHEA) enanthate, comprising:
placing a portion of unprocessed DHEA into a solvent;
allowing the portion of unprocessed DHEA to dissolve completely into the solvent, thereby forming a solution;
mixing a first fluid into the solution;
adding a second fluid slowly into the solution to cause a reaction of the portion of unprocessed DHEA; and
ceasing adding the second fluid once substantially all of the portion has been reacted.

2. The method of claim 1, wherein the solvent comprises Benzene.

3. The method of claim 1, wherein the first fluid comprises Pyridine.

4. The method of claim 1, wherein the second fluid comprises Heptanoyl Chloride.

5. The method of claim 1, wherein the portion of unprocessed DHEA comprises unprocessed 5-DHEA.

6. The method of claim 1, wherein the portion of unprocessed DHEA comprises unprocessed 1-DHEA.

7. The method of claim 1, wherein the portion of unprocessed DHEA comprises unprocessed 4-DHEA.

8. The method of claim 1, further comprising administering an effective amount of the DHEA enanthate to a human so as to improve at least one of adrenal hormonal balance, improved immune system function, reduction of adipose tissue, skeletal muscle growth, reduction of abdominal bloating, improved recovery from training, increased strength, and increased endurance.

9. The method of claim 8, wherein administering is selected from the group consisting of perorally, transdermally, sublingually, and instranasally.

10. The method of claim 8, wherein the effective amount is a daily dosage ranging between 10 mg and 2000 mg.

11. A method for providing dehydroepiandrosterone (DHEA) enanthate to a human so as to improve at least one of adrenal hormonal balance, improved immune system function, reduction of adipose tissue, skeletal muscle growth, reduction of abdominal bloating, improved recovery from training, increase in strength, and increased endurance, the method comprising:
dissolving a portion of unprocessed DHEA into a suitable quantity of Benzene to form a solution;
adding an effective amount of Pyridine into the solution;
causing a formation of the DHEA enanthate by slowly adding Heptanoyl Chloride into the solution until substantially all of the portion of unprocessed DHEA has been reacted; and
administering an effective amount of the DHEA enanthate to the human.

12. The method of claim 11, further comprising providing the DHEA enanthate in the form of any one of 5-DHEA enanthate, 1-DHEA enanthate, and 4-DHEA enanthate.

13. The method of claim 11, wherein administering is selected from the group consisting of perorally, transdermally, sublingually, and instranasally.
